(19) United States
(12) Patent Application Publication
(10) Pub. No.: US 2002/0111641 A1
(43) Pub. Date: Aug. 15, 2002

(54) Bioabsorbable surgical clip with engageable expansion structure

(75) Inventors: James Arthur Peterson, Edina, MN (US); Delmer L. Smith, Edina, MN (US); Lawrence E. Sloma, Fridley, MN (US); Joseph M. Gryskiewicz, Edina, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Incisive Surgical, Inc.

(21) Appl. No.: 10/041,705

(22) Filed: Jan. 8, 2002

Related U.S. Application Data
(60) Provisional application No. 60/260,289, filed on Jan. 8, 2001.
(60) Provisional application No. 60/260,324, filed on Jan. 8, 2001.
(60) Provisional application No. 60/260,252, filed on Jan. 8, 2001.

(51) Int. Cl. A61B 17/08

(52) U.S. Cl. 606/157

(57) ABSTRACT

A shape memory, bioabsorbable surgical clip is adapted to be more effectively delivered and operated by an applicator. The surgical clip is formed of a bioabsorbable material having sufficient shape memory so that when it is deformed from the low stress state it tends to return to the low stress state. The surgical clip provides expansion engagement structures adapted to be engaged by mating force applying structures incorporated in an applicator. Preferably, the surgical clip is generally C-shaped in the relaxed state. It generally includes a bending beam and two constraint segments. When the surgical clip is deformed into a stressed state the constraint segments assume a generally parallel orientation. Preferably, the expansion engagement structures may be located near the juncture between the bending beam and the two constraint segments.

### CLAIM TO PRIORITY

This application claims priority to U.S. Provisional Applications Nos. 60/260,289, 60/260,324 and 60/260,252 all filed on Jan. 8, 2001.

### FIELD OF THE INVENTION

The present invention relates generally to surgical fasteners and more particularly to bioabsorbable fasteners used for the surgical repair of tissues.

### BACKGROUND OF THE INVENTION

Conventional surgical technique makes use of sutures, staples, screws and plates to close wounds and to connect tissues. In recent years bioabsorbable materials have been adopted for some surgical fixation devices; however, simple substitution of bioabsorbable materials into the designed conventional non-absorbable closure devices does not result in a useable product.

The principal advantage of surgical fastening by clipping or stapling is the speed with which a wound or incision can be closed. This minimizes surgical time and stress on the patient. It is also helpful if the patient is non-cooperative. For example, children are often poorly cooperative with suturing procedures. Therefore, the more quickly that the procedure can be performed the better.

Surgical fasteners (including clips and staples) permit the surgeon to rapidly close a wound with a mechanical fastener that holds the tissue together while the wound heals. Such fasteners are often used to secure together skin, internal tissues and even bone. Generally, surgical fasteners are made from durable biocompatible materials. Biocompatible materials are well tolerated by the body and the immune system. They do not encourage a strong immune response. Thus, in some situations they can be left in place without creating inflammation, granuloma and scarring.

However, there are also many circumstances in which leaving a surgical fastener in place may be undesirable. Surgical stapling or clipping has typically had limited application where the staples are to be left in place after the wound has healed. Retained foreign bodies, even those of biocompatible materials are undesirable in some locations.

Removal of surgical fasteners may be readily accomplished if the devices are in or near the level of the skin. If the fasteners are located in deeper tissues removal may be difficult or impossible; removal of fasteners generally creates a new wound along with attendant scarring.

Both metallic and non-metallic surgical fasteners are in common use. Some of the non-metallic fasteners are formed from bioabsorbable resinous materials such as blends of lactide/glycolide copolymer. Plastic materials of this type are widely known and commercially available, for example, under the trade names of “POLYSORB” and “LACTOMER” plastic. Typically, fasteners made from these materials lose a substantial portion of their tensile strength after a few weeks of exposure to human tissue. Still later, after loss of much of their strength, the fasteners fragment and are metabolized by the body and therefore dissolve over time.

In certain surgical procedures it is desirable to close the skin wound with sutures lying completely in the dermis layer. This form of subcuticular suturing minimizes the occurrence of visible scarring. However, such subcuticular suturing is very tedious and is time consuming to perform. Effective absorbable clips or staples would be valuable in these circumstances.

A recent example of a bioabsorbable subcuticular fastener system is known from U.S. Pat. No. 5,618,311 to Gryskiewicz. The surgical clip of the '311 patent is spread by an applicator employing spreader pins to apply force to an inner surface of the C-shaped clip so as to open the clip. While the design represents an improvement over previous designs, there can be difficulties with the interaction between the spreader pins and the clip of this design. Consequently, there remains a continuing need to develop better bioabsorbable clips.

### SUMMARY OF THE INVENTION

The present invention advances the art of bioabsorbable surgical clips by providing a shape memory, bioabsorbable surgical clip that may be more effectively delivered and operated by an applicator. The surgical clip of the present invention is formed of a bioabsorbable material having sufficient shape memory so that when it is deformed from the low stress state it tends to return to the low stress state. The surgical clip of the present invention provides for expansion engagement structures adapted to be engaged by mating force applying structures incorporated in an applicator to enable more effective interaction between the applicator and the clip.

The surgical clip of the present invention is generally C-shaped in the relaxed state. It generally includes a bending beam and two constraint segments. When the surgical clip is deformed into a stressed state the constraint segments assume a generally parallel orientation. Preferably, the constraint segments each have a piercing segment at their ends, each piercing segment being adapted to pierce the tissue to be fastened. Preferably, the expansion engagement structures are located near the juncture between the bending beam and the two constraint segments. In the stressed state, the surgical clip is in an open-mouth configuration. In the relaxed state, the surgical slip is in a closed-mouth configuration.

### BRIEF DESCRIPTION OF THE DRAWINGS

FIG. 1 is a perspective view of an orthogonal embodiment of a clip in accordance with the present invention in relaxed, closed condition.

FIG. 2 is a perspective view of the orthogonal clip embodiment of FIG. 1 in a stressed, open condition.

FIG. 3 is a perspective view of an in-line embodiment of a clip in accordance with the present invention in a relaxed, closed condition.

FIG. 4 is a perspective view of the in-line clip embodiment of FIG. 3 in a stressed, open condition.

FIG. 5 is a perspective view of an offset embodiment of a clip in accordance with the present invention in a relaxed, closed condition.

FIG. 6 is a side view of a notched embodiment of a clip in accordance with the present invention in a relaxed, closed condition.

FIG. 7 is a perspective view of the notched clip embodiment of FIG. 6.

### DETAILED DESCRIPTION OF THE PREFERRED EMBODIMENTS

The overall system includes a clip and an applicator to deliver the clip. The complete applicator is not shown in this disclosure, however a pin-like interface element is depicted in some of the figures and described.

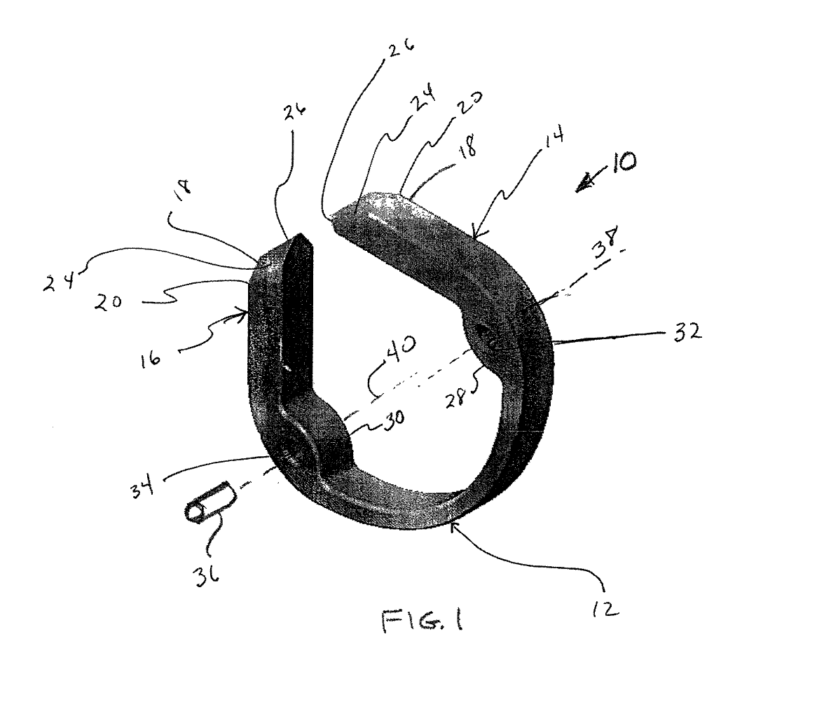

Referring to FIG. 1, orthogonal clip 10 is generally C-shaped. FIG. 1 depicts orthogonal clip 10 in a relaxed or low stress state. This is the shape that the orthogonal clip 10 assumes if not deformed. Orthogonal clip 10 is formed of a bioabsorbable material having sufficient shape memory so that when it is deformed from the low stress state it tends to return to the low stress state. Sufficient shape memory means that the clip will tend to return substantially to its original shape after having been deformed for a period of time lasting from seconds to minutes. The tendency to return to original shape provides enough force to hold the tissue together and to continue to apply a force in that direction if the partially closed orthogonal clip 10 does not achieve the fully closed position.

For example, appropriate bioabsorbable materials include bioabsorbable resinous materials such as blends of lactic acid/glycolide copolymer. Plastic materials of this type are widely known and commercially available, for example, under the trade names of “POLYSORB”and “LACTOMER” plastic.

Orthogonal clip 10 is generally bilaterally symmetrical in this disclosure though a somewhat asymmetrical structure may be utilized in some circumstances. It is specifically contemplated that the surgical clips of the present invention include asymmetrical embodiments as well as symmetrical embodiments. Further, the clips of the present invention may include embodiments that combine features of the several embodiments of the invention disclosed herein. Orthogonal clip 10 generally presents three regions. These are bending beam 12, first constraint segment 14, and second constraint segment 16.

Bending beam 12 is centrally located between first constraint segment 14 and second constraint segment 16. Bending beam 12 is preferably curved outwardly when in the relaxed state. Bending beam 12 is preferably rectangular in cross-section and of generally consistent cross-section along its length. It is designed to deform while keeping itself and first constraint segment 14 and second constraint segment 16 in generally planar alignment.

First constraint segment 14 and second constraint segment 16 are generally mirror images of one another but may also be asymmetrical. Each presents at its end 18 a piercing segment: first piercing segment 20 and second piercing segment 22, respectively. Each of first piercing segment 20 and second piercing segment 22 may include beveled surfaces 24 defining cutting edges 26. First piercing segment 20 and second piercing segment 22 are desirably sharp enough to pierce whatever tissue is to be secured. First constraint segment 14 and second constraint segment 16 are preferably sufficiently rigid to constrain the tissue. Constraint segments 14, 16 and equivalent parts of the following embodiments may be adapted to grasp and pull together pierced tissue to be joined as well as to clamp together separate pieces of tissue without piercing them.

Orthogonal clip 10 also presents reinforcing bosses 28, 30 defining first bore 32 and second bore 34. Reinforcing bosses 28, 30 are preferably located at the juncture of bending beam 12 and each of first constraint segment 14 and second constraint segment 16. First bore 32 and second bore 34 are each adapted to receive a pin 36. Pins 36 are a part of applicator (not shown). While pins 36 and respective bores 32, 34 are preferably identical in mating operation, it will be understood that bores 32 and 34 and their corresponding pins 36 could each be of different sizes and configurations.

As will be apparent from the structure of orthogonal clip 10 and the following embodiments of the invention, the engagement structure represented by first bore 32 and second bore 34 may take many different forms. The engagement structure allows a mating force application member to apply force to the clip 10 to urge it to an open position to facilitate use. Engagement structures may include pins, bores, apertures, knobs and grips.

Applicator (not shown) may take many different forms. As will be apparent from this disclosure, the applicator serves to apply a force to spread the various embodiments of the invention into an open configuration so as to configure the clip to surround the desired tissue and to facilitate placement. The force application members of the applicator serve to engage with the engagement structures of the invention and allow the application of spreading force. Applicators may employ force application members that include but are not limited to pins, springs, apertures, and any other structure capable of mating with an engagement structure. Applicators may function manually or automatically. A simple, exemplary applicator resembles a hemostat with force application members at the end of each arm.

First bore 32 and second bore 34 each present a first and second bore axis 38, 40 oriented generally orthogonal to the plane in which bending beam 12, first constraint segment 14 and second constraint segment 16 of orthogonal clip 10 all lie. First bore axis 38 and second bore axis 40 are generally parallel to each other whether the orthogonal clip 10 is in the relaxed or stressed state. Bores 32 and 34 are representative of a variety of apertures that can be employed to allow engagement members of an applicator (not shown) to engage the surgical clip of this and the following embodiments of the invention.

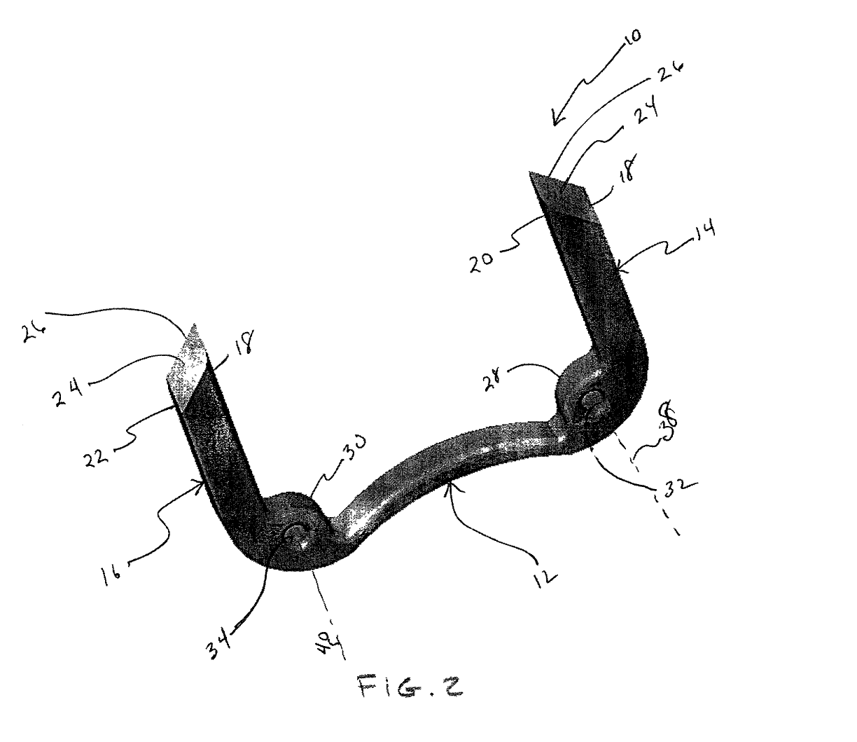

In operation, applicator (not shown) applies force to first bore 32 and second bore 34 via pins 36 to deform orthogonal clip 10 from the relaxed state depicted in FIG. 1 to the stressed state depicted in FIG. 2. In an alternate embodiment (not shown), it will be understood that an anvil arrangement could be used on either the interior or exterior surface of the bending beam 12 to assist in deforming clip 10 from the relaxed state to the stressed state. As can be seen in FIG. 2, in the stressed state first constraint segment 14 and second constraint segment 16 are generally parallel and are optimally oriented for insertion into the tissue that the surgeon desires to clip together. Once orthogonal clip 10 is in the desired position and first constraint segment 14 and second constraint segment 16 are embedded on the desired tissue, the surgeon releases applicator (not shown) and removes pins 36 from first bore 32 and second bore 34. As orthogonal clip 10 returns to the relaxed state, first constraint segment 14 and second constraint segment 16 pull the tissue together and bring the wound edges into contact with each other to allow healing. Because orthogonal clip 10 is made of bioabsorbable material, it is broken down and absorbed by the body over a period of time.

In the stressed state, the orthogonal clip 10 and the following embodiments of the present invention present an open “mouth” into which tissue to be joined may be placed. In the relaxed state, the surgical clips of the present invention present a closed-mouth that presses the tissue to be joined together to facilitate healing.

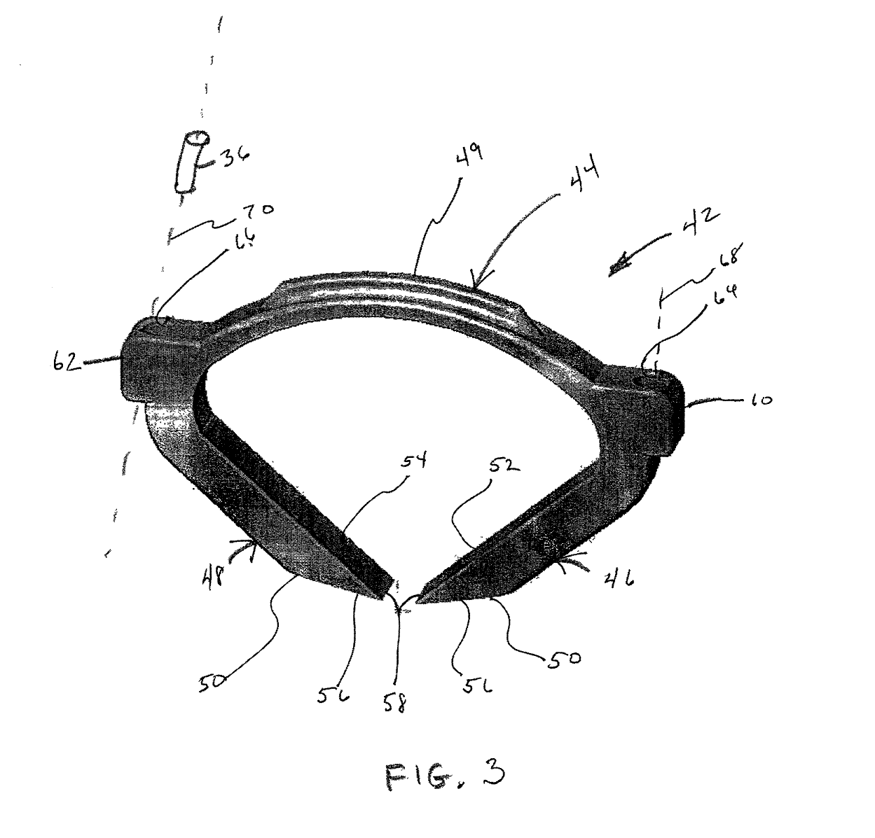

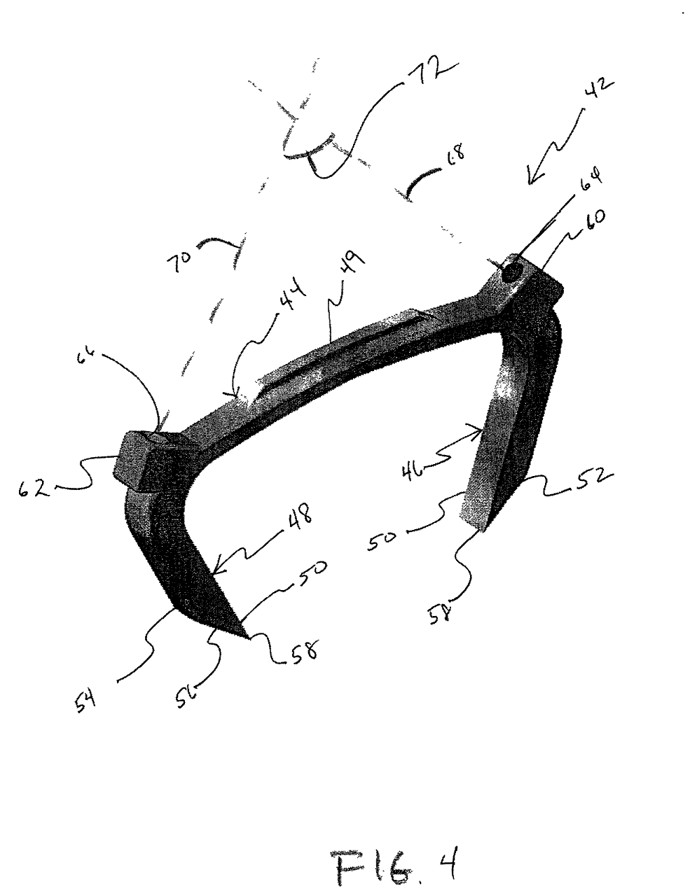

Referring to FIGS. 3 and 4, another embodiment of the invention is generally C-shaped. FIG. 3 depicts in-line clip 42 in a relaxed or low stress state. FIG. 4 depicts this embodiment in the stressed state. In-line clip 42 is formed of a bioabsorbable material similar to that of orthogonal clip 10. In-line clip 42 is generally bilaterally symmetrical, though a somewhat asymmetrical structure may be utilized in some circumstances. In-line clip 42 generally presents three regions. These are bending beam 44, first constraint segment 46 and second constraint segment 48.

Bending beam 44 is centrally located between first constraint segment 46 and second constraint segment 48. In this embodiment, bending beam 44 is preferably curved outwardly when in the relaxed state and presents ridge 49 on the outside thereof. Bending beam 44 is preferably T-shaped in cross-section for part of its length and generally rectangular in cross-section for the remainder of its length. It is designed to deform while keeping itself and first constraint segment 46 and second constraint segment 48 in generally planar alignment. One skilled in the art will appreciate that alteration of the size and shape of ridge 49 alters the force with which bending beam 44 returns to the unstressed condition.

First constraint segment 46 and second constraint segment 48 are generally mirror images of one another. Each presents at its end 50 a piercing segment: first piercing segment 20 and second piercing segment 22, respectively. Each of first piercing segment 52 and second piercing segment 54 may include beveled surfaces 56 defining cutting edges 58. First piercing segment 52 and second piercing segment 54 are desirably sharp enough to pierce whatever tissue is to be secured. First constraint segment 46 and second constraint segment 48 are preferably sufficiently rigid to constrain the tissue.

In-line clip 42 also presents first and second application lugs 60, 62 defining first bore 64 and second bore 66. First application lug 60 and second application lug 62 are preferably located at the outside of the juncture of bending beam 44 and each of first constraint segment 46 and second constraint segment 48. First application lug 60 and second application lug 62 define a first bore 64 and a second bore 66, respectively. First bore 64 and second bore 66 are each adapted to receive pin 36. Pins 36 are part of applicator (not shown). First bore 64 and second bore 66 each present a first and second bore axis 68, 70 oriented generally parallel to each other when in-line clip 42 is in the relaxed state. First bore axis 68 and second bore axis 70 are preferably oriented in the plane in which bending beam 44, first constraint segment 46 and second constraint segment 48 all lie. When in-line clip 42 is in the stressed state, first bore axis 68 and second bore axis 70 intersect at an intersection angle 72.

In operation, a surgeon using applicator (not shown) applies force to first bore 64 and second bore 66 via pins 36 to deform in-line clip 42 from the relaxed state depicted in FIG. 3 to the stressed state depicted in FIG. 4. As can be seen in FIG. 4, in the stressed state first constraint segment 46 and second constraint segment 48 are generally parallel and are optimally oriented for insertion into the tissue that the surgeon desires to clip together. Note that as in-line clip 42 is forced from the relaxed state to the stressed state, first bore axis 68 and second bore axis 70 go from a generally parallel orientation to intersect at intersection angle 72.

Once in-line clip 42 is in the desired position and first constraint segment 46 and second constraint segment 48 are embedded on the desired tissue, the surgeon releases applicator (not shown) and then removes pins 36 from first bore 64 and second bore 66. As in-line clip 42 returns to the relaxed state, first constraint segment 46 and second constraint segment 48 pull the tissue together and bring the wound edges into contact with each other to allow healing. Because in-line clip 42 is made of bioabsorbable material it is broken down and absorbed by the body over a period of time.

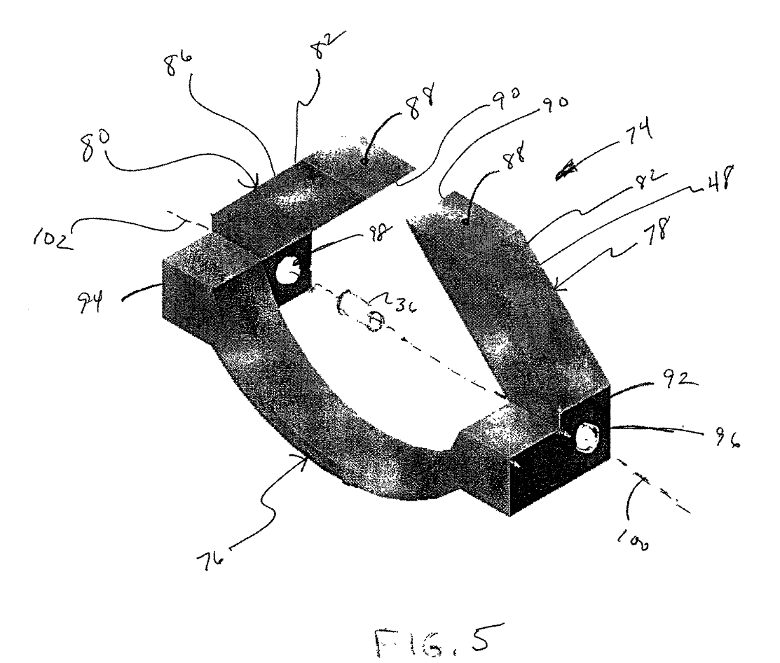

Referring to FIG. 5, another embodiment of the invention is depicted. Offset clip 74 generally includes offset bending beam 76, first constraint segment 78, and second constraint segment 80. This embodiment of the invention is generally C-shaped but offset bending beam 76 is offset from first constraint segment 78 and second constraint segment 80. FIG. 5 depicts an offset clip 74 in a relaxed or low stress state. Offset clip 74 is formed of a dissolvable material similar to that of orthogonal clip 10 and in-line clip 42. Offset clip 74 is generally bilaterally symmetrical, though a somewhat asymmetrical structure may be utilized in some circumstances.

Offset bending beam 76 is centrally located between first constraint segment 78 and second constraint segment 82. In this embodiment, bending beam 76 is preferably curved outwardly when in the relaxed state. Offset bending beam 76 is, preferably, generally rectangular in cross-section. It is designed to deform while keeping itself and first constraint segment 78 and second constraint segment 80 in generally parallel alignment with offset bending beam 76 in a first plane and first constraint segment 78 and second constraint segment 80 in a second plane parallel to the first.

First constraint segment 78 and second constraint segment 80 are generally mirror images of one another. Each presents at its end 82 a piercing segment: first piercing segment 84 and second piercing segment 86, respectively. Each of first piercing segment 84 and second piercing segment 86 may include beveled surfaces 88 defining cutting edges 90. First piercing segment 84 and second piercing segment 86 are desirably sharp enough to pierce whatever tissue is to be secured. First constraint segment 78 and second constraint segment 80 are preferably sufficiently rigid to constrain the desired tissue.

Offset clip 74 also presents first and second offset lugs 92, 94 defining first bore 96, and second bore 98. First offset lug 92 and second offset lug 94 are preferably located at the juncture of bending beam 76 and each of first constraint segment 78 and second constraint segment 80 and interconnect bending beam 76 with first constraint segment 78 and second constraint segment 80. First offset lug 78 and second offset lug 80 define a first bore 96 and a second bore 98, respectively. First bore 96 and second bore 98 are each adapted to receive pin 36. Pin 36 is a part of applicator (not shown). First bore 96 and second bore 98 each present a first and second bore axis 100, 102 oriented generally coaxial to each other when in offset clip 74 is in the relaxed state. First bore axis 100 and second bore axis 102 are preferably oriented in the plane in which first constraint segment 78 and second constraint segment 80 lie. When offset clip 74 is in the stressed state, first bore axis 100 and second bore axis 102 intersect at an intersection angle (not shown).

In operation, a surgeon using applicator (not shown) applies force to first bore 96 and second bore 98 via pins 36 to deform offset clip 74 from the relaxed state depicted in FIG. 5 to the stressed state (not shown). In the stressed state, first constraint segment 78 and second constraint segment 80 are generally parallel and are optimally oriented for insertion into the tissue that the surgeon desires to fasten together. Note that as offset clip 74 is forced from the relaxed state to the stressed state bore axis 100 and bore axis 102 go from a generally coaxial orientation to intersect at an intersection angle (not shown).

Once the offset clip 74 is in the desired position and first constraint segment 78 and second constraint segment 80 are embedded on the desired tissue, the surgeon releases applicator (not shown) and then removes pins 36 from first bore 96 and second bore 98. As the offset clip 74 returns to the relaxed state, first constraint segment 78 and second constraint segment 80 pull the tissue together and bring the wound edges into contact with each other to allow healing. Because the offset clip 74 is made of bioabsorbable material, it is broken down and absorbed by the body over a period of time.

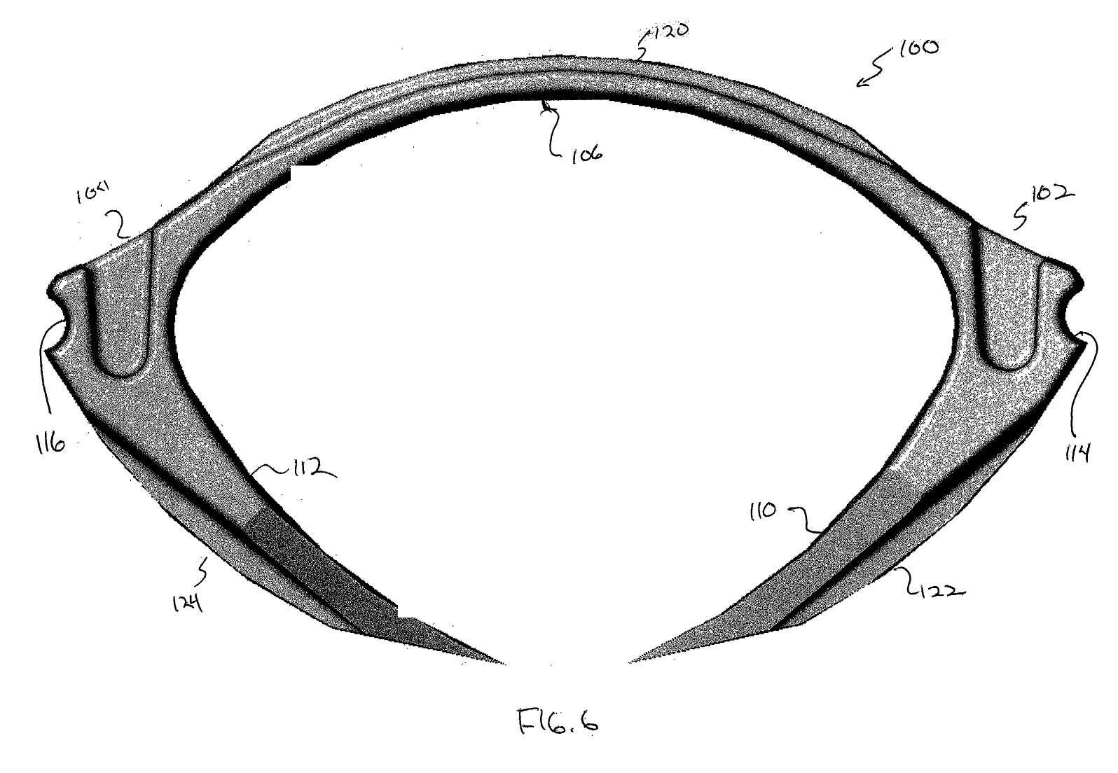

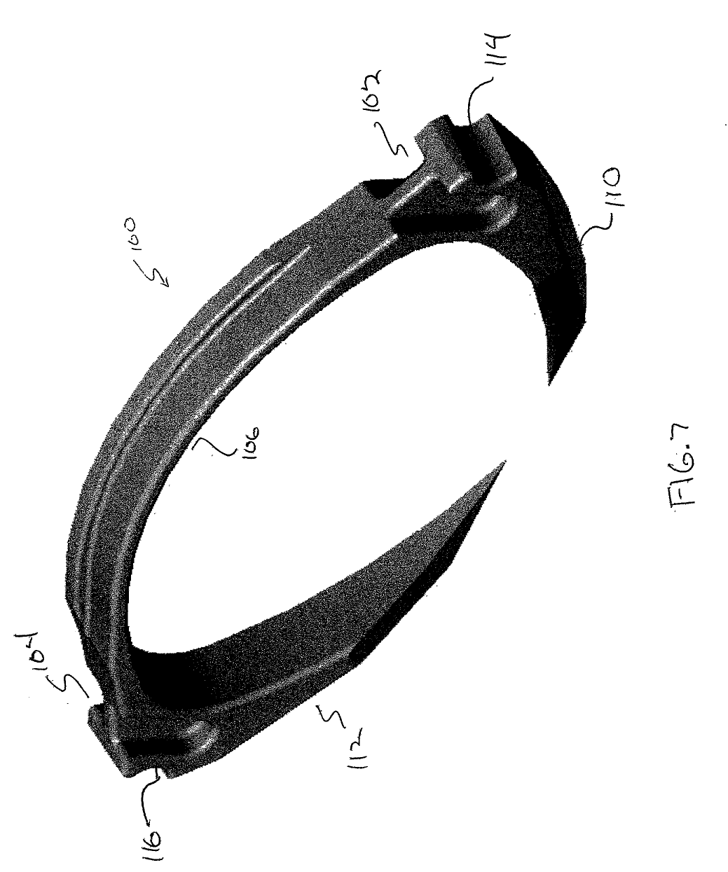

Referring to FIGS. 6 and 7, a notched embodiment of clip 100 is shown. Notches 102 and 104 provide the expansion engaging structures on notched clip 100 that mate with and interact with the force application members of an applicator (not shown) for opening the notched clip from a relaxed position to a stressed position. Notched clip 100 includes a backspan member 106 and piercing/constraining segments 110, 112 that operate in a manner as previously described with respect to the other embodiments. In this embodiment, additional pivot notches 114, 116 are provided proximate notches 102, 104 to assist in the application of a rotational force to notched clip 100 so as to move segments 110 and 112 from a relaxed state to a stressed state. It will be apparent that notches 102 and 104 can assume various configurations on one or both sides of clip 100 and various shapes, depending upon the mating and interaction desired with a given applicator. Ribs 120 along backspan 106 and ribs 122 and 124 along segments 110 and 112 may also be provided for additional strength and/or to improve the stacking or packaging of the notched clips 110.

The present invention may be embodied in other specific forms without departing from the spirit of the essential attributes thereof; therefore, the illustrated embodiments should be considered in all respects as illustrative and not restrictive, reference being made to the appended claims rather than to the foregoing description to indicate the scope of the invention.

What is claimed is:

1. A surgical clip for retaining tissue and for delivery by an applicator of type having one or more force application members, the clip comprising:
a bending beam having two ends and a sufficient shape memory to generally return to a relaxed position and to hold the tissue together after being forcibly distorted to a stressed position;
at least two constraint segments having one end thereof operably coupled to a respective end of the bending beam, the constraint segments being sufficiently rigid to retain the tissue when the bending beam returns to the relaxed position; and
the clip presenting at least two expansion engagement structures adapted for engaging the force application members such that the force application members of the applicator may be used to apply a force to the clip to distort it from the relaxed position to the stressed position; and
wherein the surgical clip is formed substantially of a bioabsorbable material, whereby the surgical clip is absorbed by a living tissue over a period of time.

2. The surgical clip of claim 1, the constraint segments each comprising a piercing portion sufficiently sharp to pierce the tissue.

3. The surgical clip of claim 1, in which the bending beam has a generally curved shape when in the relaxed position.

4. The surgical clip of claim 1, in which the engagement structures comprise bores into the clip and the force application members comprise pins.

5. The surgical clip of claim 4, the bores each having an axis, the constraint segments lying in a common plane in which the axis of the bores is generally orthogonal to the plane.

6. The surgical clip of claim 4, the bores each having an axis, the constraint segments lying in a common plane in which the axis of the bores is generally parallel to the plane.

7. The surgical clip of claim 4, the bores each having an axis, the constraint segments lying in a common plane in which the axes of the bores is generally coincident with the plane.

8. The surgical clip of claim 4, the bores each having an axis, in which the axes of the bores are coaxial with one another when the clip is in the relaxed state.

9. The surgical clip of claim 1, further comprising a ridge on the bending beam.

10. The surgical clip of claim 1, in which the bending beam is offset from the two constraint segments.

11. The surgical clip of claim 1, in which the engagement structures are proximate the juncture between the bending beam and the constraint segments.

12. The surgical clip of claim 1, in which the engagement structures are at a fixed location on the surgical clip.

13. A method for surgically joining living tissue, the method comprising the steps of:
applying force application members of an applicator to a surgical clip, the clip being formed from a bioabsorbable material and having engagement structures adapted to mate with the force application members;
forcing the surgical clip to assume a stressed, open configuration with the applicator;
enveloping the living tissue within the surgical clip;
relaxing the force on the surgical clip such that the surgical clip may elastically return to an unstressed state whereby the living tissue is pressed together; and
disengaging the force application members of the applicator from the surgical clip.

14. The method of claim 13, further comprising the step of piercing the living tissue with a piercing portion of the surgical clip.

15. A surgical clip for retaining tissue and for delivery by an applicator of type having one or more force application members, the clip comprising:
means for resiliently returning to a relaxed position after deformation having two ends and a sufficient shape memory to return to a relaxed position after being forcibly distorted to a stressed position;
at least two means for constraining tissue coupled at each end of the resilient return means, the constraining means being sufficiently rigid to retain the tissue when the resilient return means returns to the relaxed position;
the clip presenting at least two means for mating with the force application members such that the applicator may be used to apply a force to the clip to distort it from the relaxed position to the stressed position; and
wherein the surgical clip is formed substantially of a bioabsorbable material, whereby the surgical clip is absorbed by a living tissue over a period of time.

16. The surgical clip of claim 15, the constraining means each comprising a piercing portion sufficiently sharp to pierce the tissue.

17. The surgical clip of claim 15, in which the receiving means comprise a bore into the clip and the engagement member comprises a pin.

18. The surgical clip of claim 17, the bores each having an axis, the constraining means lying in a common plane in which the axis of the bores is generally orthogonal to the plane.

19. The surgical clip of claim 17, the bores each having an axis, the constraining means lying in a common plane in which the axis of the bores is generally parallel to the plane.

20. The surgical clip of claim 17, the bores each having an axis, the constraining means lying in a common plane in which the axes of the bores is generally coincident with the plane.

21. The surgical clip of claim 17, the bores each having an axis, in which the axes of the bores are coaxial with one another when the clip is in the relaxed state.

22. The surgical clip of claim 15, further comprising a ridge on the resilient return means.

23. The surgical clip of claim 15, in which the resilient return means has a generally curved shape when in the relaxed position.

24. A surgical clip for retaining tissue and for delivery by an applicator of type having one or more force application members, the clip comprising:
a bending beam having two ends and a sufficient shape memory generally return to a relaxed position and to hold the tissue together after being forcibly distorted to a stressed position;
at least two constraint segments having one end thereof operably coupled to a respective end of the bending beam, the constraint segments being sufficiently rigid to retain the tissue when the bending beam returns to the relaxed position;
the clip presenting at least two expansion engagement structures on fixed locations on the clip adapted to engage the force application members such that the force application members of the applicator may be used to apply a force to the clip to distort it from the relaxed position to the stressed position; and
wherein the surgical clip is formed substantially of a bioabsorbable material, whereby the surgical clip is absorbed by a living tissue over a period of time.

25. The surgical clip of claim 24, the constraint segments each comprising a piercing portion sufficiently sharp to pierce the tissue.

26. The surgical clip of claim 24, in which the bending beam has a generally curved shape when in the relaxed position.

27. The surgical clip of claim 24, in which the engagement structures comprise bores into the clip and the force application members comprise pins.

28. The surgical clip of claim 27, the bores each having an axis, the constraint segments lying in a common plane in which the axis of the bores is generally orthogonal to the plane.

29. The surgical clip of claim 27, the bores each having an axis, the constraint segments lying in a common plane in which the axis of the bores is generally parallel to the plane.

30. The surgical clip of claim 27, the bores each having an axis, the constraint segments lying in a common plane in which the axes of the bores is generally coincident with the plane.

31. The surgical clip of claim 27, the bores each having an axis, in which the axes of the bores are coaxial with one another when the clip is in the relaxed state.

32. The surgical clip of claim 24, further comprising a ridge on the bending beam.

33. The surgical clip of claim 24, in which the bending beam is offset from the two constraint segments.

34. The surgical clip of claim 24, in which the engagement structures are proximate the juncture between the bending beam and the constraint segments.

35. A surgical clip for retaining tissue and for delivery by an applicator of type having one or more force application members, the clip comprising:
a bending beam having two ends and a sufficient shape memory generally return to a relaxed position and to hold the tissue together after being forcibly distorted to a stressed position;
at least two constraint segments having one end thereof operably coupled to a respective end of the bending beam, the constraint segments being sufficiently rigid to retain the tissue when the bending beam returns to the relaxed position;
the clip presenting at least two expansion engagement structures located proximate the ends of the bending beam and proximate the ends of the constraint segments, the expansion engagement structures adapted to mate with the force application members such that the force application members of the applicator may be used to apply a force to the clip to distort it from the relaxed position to the stressed position; and
wherein the surgical clip is formed substantially of a bioabsorbable material, whereby the surgical clip is absorbed by a living tissue over a period of time.

36. The surgical clip of claim 35, the constraint segments each comprising a piercing portion sufficiently sharp to pierce the tissue.

37. The surgical clip of claim 35, in which the bending beam has a generally curved shape when in the relaxed position.

38. The surgical clip of claim 35, in which the engagement structures comprise bores into the clip and the force application members comprise pins.

39. The surgical clip of claim 37, the bores each having an axis, the constraint segments lying in a common plane in which the axis of the bores is generally orthogonal to the plane.

40. The surgical clip of claim 37, the bores each having an axis, the constraint segments lying in a common plane in which the axis of the bores is generally parallel to the plane.

41. The surgical clip of claim 37, the bores each having an axis, the constraint segments lying in a common plane in which the axes of the bores is generally coincident with the plane.

42. The surgical clip of claim 37, the bores each having an axis, in which the axes of the bores are coaxial with one another when the clip is in the relaxed state.

43. The surgical clip of claim 35, further comprising a ridge on the bending beam.

44. The surgical clip of claim 35, in which the bending beam is offset from the two constraint segments.
